(19) United States
(12) Patent Application Publication
(10) Pub. No.: US 2023/0245734 A1
(43) Pub. Date: Aug. 3, 2023

(54) AUTOMATIC ENROLLMENT WITH A SERVICE FOR A MEDICAL DEVICE

(71) Applicant: Medtronic, Inc., Minneapolis, MN (US)

(72) Inventors: Matthew R. Yoder, Crystal, MN (US); Eric D. Dorphy, MapteGrove, MN (US)

(21) Appl. No.: 17/649,325

(22) Filed: Jan. 28, 2022

(51) Int. Cl. G16H 10/60; G16H 50/20

(52) U.S. Cl. CPC G16H 10/60; G16H 50/20

(57) ABSTRACT

This disclosure is directed to systems and techniques operative to, responsive to a receiving input comprising a globally unique identifier (GUID), generate a completed enrollment request for communication, via the communication circuitry, to the computing service to bring an implanted medical device into service for a patient, wherein the computing service stores data comprising a mapping between the GUID and attribute information for the completed enrollment request; and receive from the computing service a successful enrollment response for the implanted medical device.

### FIELD

The disclosure relates generally to medical systems and, more particularly, medical systems in which an application is used by a patient with a medical device.

### BACKGROUND

Medical systems may monitor various types of data of a patient or a group of patients for one or several purposes. Amongst the numerous examples, some medical systems may record measurements of a patient and their heart as indicia of cardiac health for that patient, which may be memorialized in raw data and/or processed data formats; as one example, electric signals representing cardiac activity over a period of time may be memorialized as a cardiac electrogram (EGM), and then processed into other indicia of the cardiac health of the patient. In some examples, the medical system may monitor the cardiac EGM to detect one or more types of arrhythmia, such as bradycardia, tachycardia, fibrillation, or asystole (e.g., caused by sinus pause or AV block).

Medical professionals may use the medical system on their patients for a number of reasons such as having the medical system record patient data for future use. Some medical professionals rely on that data to provide their patients with the best medical care. For various purposes, a medical professional may program the medical system to operate as desired, which may be in accordance with a certain algorithm, and calibrate medical system components to detect health events, deliver a therapy, and so forth. In some examples, the medical system may include one or more of an implantable medical device or a wearable device to collect various measurements used to detect changes in patient cardiac health. In some examples, an implantable or a wearable medical device may be configured with an algorithm for monitoring patient cardiac activity for cardiac events. In some examples, the patient may interact with an application, e.g., on a mobile device of the patient, associated with their medical device. The application may collect information, e.g., symptom information, from a patient, provide information to the patient, facilitate communication between the medical device and a computing service, or facilitate communication between the patient and a clinician, e.g., regarding the medical device, as examples.

### SUMMARY

Medical systems and techniques as described herein automatically enroll a patient and the patient's instance of an application for a medical device into a computing service at or around a time that medical device is affixed to, e.g., implanted in, a patient. In general, there typically is considerable latency between the patient receiving the medical device and successfully enrolling themself and the application into the computing service for remote monitoring, health event detection, and overall medical device support. Enrollment may be required prior to bringing that medical device online (e.g., for exchanging data with the computing service) and/or into operation; however, even when enrollment is not required to commence medical device operation, there is great difficulty put on the patient for completing an enrollment process (e.g., including new account registration). By immediately/quickly completing the enrollment process to satisfaction of computing service, the patient receives, without substantially delay, the medical care they were intended to receive.

The computing service of the present disclosure generally provides an implanted medical device and/or an instantiated medical application with access to various resources. The implanted medical device and/or the instantiated medical application may use these resources to support their native functionality, which may include detecting changes in the patient's health that correlate to changes in the patient data (e.g., health events). Furthermore, the computing service may provide resources that the implanted medical device and/or the instantiated medical application can leverage to enhance their native functionality, for example, with accurate predictions regarding health events (including fewer false detections), detailed patient data with a reduced error rate in its collection, and better resolution into the patient's physiological history over time. The computing service may enhance current medical device/medical application operations with additional aspects of the patient's health to analyze for evidence of health events.

The computing service may interact with the patient immediately upon implantation, for instance, to provide data, via the medical application (or simply “application”) regarding that patient's health. The patient, via the application, may migrate various datasets of patient information (of which a portion is provided by the implanted medical device) to the computing service for storage, processing, and possibly, distribution to approved entities (e.g. a hospital or a clinician). There are other ways for the computing service to provide more insight into the patient's health by way of automatic enrollment. In total, the computing service lowers a medical device's overall resource utilization, allowing for medical devices with fewer resource capacities, such that a medical device equipped with substantial resource capacities is no longer needed for the medical systems and the techniques described herein.

In a typical enrollment process, the computing service may prescribe a set of requirements. As one pre-requisite, enrollment may entail a new (patient) account registration of an associated instance of the application and/or the associated medical device. There may be a sequence of operations in which various data attributes are exchanged with the computing service for (e.g., account) authorization in using the application (e.g., a mobile application) as an interface. The computing service, via email or another communication medium, provides identity information (e.g., a globally unique identifier) for the patient to use with the application, for example, for accessing computing resources, executing application functionality, and initiating medical device operation. There may be functionality specific to the medical device that the application and/or the computing service can enable by way of a successful enrollment and registration of the new account. By automating the enrollment process including the new account registration, the medical systems and the techniques described herein effectuate that functionality as soon as possible upon implantation of the medical device.

Therefore, having medical systems automate enrollment of new or recent implanted medical device mitigate or eliminate altogether the problems associated with other approaches, such as a tendency for false positives and false negatives and a latency between the medical implantation and the successful enrollment. In view of this and other benefits, the present disclosure describes a technological improvement in form of a technical solution that is integrated into a practical application. Unless otherwise noted, the following description references the above-mentioned medical application instance in discussion of any application.

In one example, a computing device comprises: communication circuitry communicatively coupled to a computing service via a network connection; processing circuitry; and memory comprising programming instructions that, when executed by the processing circuitry, cause the processing circuitry to: responsive to receiving input comprising a globally unique identifier (GUID) corresponding to an application for a patient having an implanted medical device, generate a completed enrollment request for communication, via the communication circuitry, to the computing service to enroll the patient with the computing service, wherein the computing service stores data comprising a mapping between the GUID and attribute information for the completed enrollment request; receive from the computing service a successful enrollment response based upon the communication of the completed enrollment request; and execute functionality of the application associated with the implantable medical device and the computing service.

In another example, a method of a medical system performed by processing circuitry of a computing device of a patient comprises: establishing a network connection for coupling communication circuitry of the computing device with a computing service; responsive to a receiving input comprising a globally unique identifier (GUID) corresponding to an application for a patient having an implanted medical device, generating a completed enrollment request for communication, via the communication circuitry, to the computing service to enroll the patient with the computing service, wherein the computing service stores data comprising a mapping between the GUID and attribute information for the completed enrollment request; receiving from the computing service a successful enrollment response based upon the communication of the completed enrollment request; and execute functionality of the application associated with the implantable medical device and the computing service.

In another example, a non-transitory computer-readable storage medium comprising program instructions that, when executed by processing circuitry of a medical device, cause the processing circuitry to: establish a network connection for coupling communication circuitry of a computing device of a patient with a computing service; responsive to a receiving input comprising a globally unique identifier (GUID) corresponding to an application for the patient having an implanted medical device, generate a completed enrollment request for communication, via communication circuitry, to a computing service to enroll the patient with the computing service, wherein the computing service stores data comprising a mapping between the GUID and attribute information for the completed enrollment request; and receive from the computing service a successful enrollment response based upon the communication of the completed enrollment request; and execute functionality of an application associated with the implantable medical device and the computing service.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1 illustrates example environment of an example medical system in conjunction with a patient, in accordance with one or more examples of the present disclosure.

FIG. 2 is a block diagram illustrating an example configuration of a medical device, in accordance with one or more examples of the present disclosure.

FIG. 3 is a conceptual side-view diagram illustrating an example configuration of the 1 MB of FIGS. 1 and 2, in accordance with one or more examples of the present disclosure.

FIG. 4 is a block diagram illustrating an example configuration of the external device of FIG. 1, in accordance with one or more examples of the present disclosure.

FIG. 5 is a block diagram illustrating an example system that includes an access point, a network, external computing devices, such as a server, and one or more other computing devices, which may be coupled to the medical device and external device of FIGS. 1-4, in accordance with one or more examples of the present disclosure.

FIG. 6 is a flow diagram illustrating an example operation for automatically enrolling a medical device with a computing service, in accordance with one or more examples of the present disclosure.

Like reference characters denote like elements throughout the description and figures.

### DETAILED DESCRIPTION

In general, medical systems according to this disclosure implement techniques for facilitating an enrollment process with a computing service for access to various resources used in detecting changes in patient health. A patient may be instantly enrolled into the computing service, for instance, by automatically registering a new account for a medical device and/or a medical application as described herein. In some examples, a medical system may implement a technique for substantially automating the enrollment process, for instance, to commence normal operation of the medical device.

A variety of medical devices (e.g., implantable devices, wearable devices, etc.) may be configured to monitor various patient data and detect changes in the patient's health that correlate to changes in the patient data (e.g., health events). A variety of medical applications (e.g., a mobile application, a desktop application, a browser application, a web application to run on a browser application, etc.) may be configured to support, in various forms, the patient data monitoring and health event detection. The present disclosure may refer to the computing service as a monitoring service for remote patient health monitoring and health event detection. In one example, the medical application may be a client application for the monitoring service. The medical application may be proprietary software code for controlling operation of the medical device.

The medical application, in general, may generate an interface to facilitate an exchange of various data between the monitoring service and a patient device (e.g., the patient's personal device such as a mobile device). Some medical applications may be configured with functionality specific to the monitoring service, whereas other medical applications may be independent (i.e., a 3rd party) of the monitoring service and therefore, capable of other functionality. While the techniques described herein may any automatically enroll any medical application instance and/or any medical device with the monitoring service, the following description may refer to the medical systems that enable automatic enrollment of medical application instances and/or implanted medical devices.

The medical devices described herein provide medical care in some form; amongst the numerous medical devices envisioned by the present disclosure, insertable cardiac monitors (e.g., LINQ & LINQ II), pacemakers, and defibrillators are illustrative examples, namely, for operating with Medtronic CareLink® online/cloud computing service for remote monitoring, health event detection, and device support. Any given medical device may communicate the patient data to the patient device and other external devices, such as the monitoring service, via the patient device. The medical application running on the patient device may associate the patient data with their (new) account. By doing so, the medical application and the monitoring service may coordinate the remote monitoring, health event detection, and device support given to the medical device. As one benefit, the monitoring service may analyze the patient data for providing a report regarding the patient's activities and health. This report may be used to inform the patient or caregiver of an important aspect of the patient's activities and health. In this manner, the techniques of this disclosure may advantageously enable improved accuracy in the detection of changes in patient health and, consequently, better evaluation of the condition of the patient.

As one example, the medical device may be a pacemaker that is implanted in the patient, and at or around the time of implantation, a smart mobile phone owned by the patient may launch a mobile application to execute the enrollment process, which may include the registration of the new patient account. The mobile application may be acquired via any available mechanism, such as by data transfer from an application repository (e.g., a property known as an APP STORE under any mobile operating system), download (e.g., via a mobile browser application), or by human activity prior to any implantation (e.g., from a storage device). The mobile application may be acquired upon implantation or may be pre-installed on the patient device.

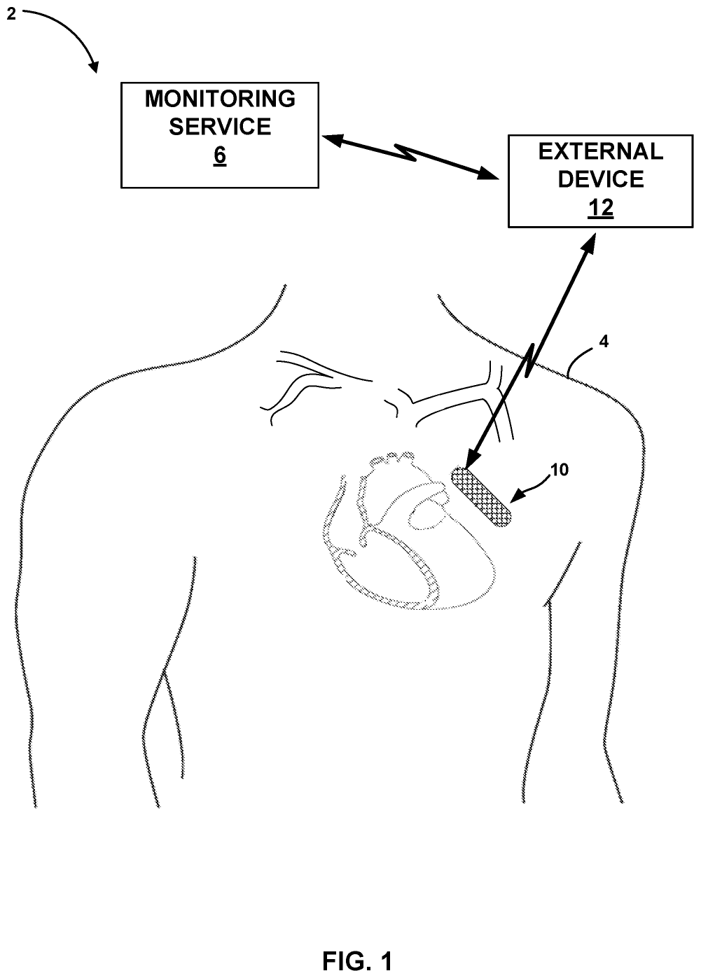

FIG. 1 illustrates the environment of an example medical system 2 in conjunction with a patient 4, in accordance with one or more techniques of this disclosure. The example techniques may be used with an implanted medical device (IMD) 10, which may be in wireless communication (via a wireless channel) with at least one of external device 12 and other devices not pictured in FIG. 1. In some examples, IMD 10 is implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette. IMD 10 includes a plurality of electrodes (not shown in FIG. 1), and is configured to sense a cardiac EGM via the plurality of electrodes. In some examples, IMD 10 takes the form of the LINQ™ ICM available from Medtronic, Inc. of Minneapolis, Minn.

External device 12 may be a computing device with a display viewable by the user and an interface for receiving user input to external device 12. In some examples, external device 12 may be a notebook computer, tablet computer, workstation, one or more servers, cellular/mobile phone, personal digital assistant, or another computing device that may run an application that enables the computing device to interact with IMD 10. One example embodiment of external device may be a personal computing device of patient 4 (e.g., a patient computing device or simply “patient device”). The user of external device 12 may be any person authorized by patient 4, such as a clinician programmer. Accordingly, another example embodiment may be a computing device for the clinician programmer.

External device 12 is configured to communicate with IMD 10 and, optionally, another computing device (not illustrated in FIG. 1), over the wireless channel or, alternatively, via a network connection from a remote location to patient 4. External device 12, for example, may communicate via near-field communication technologies (e.g., inductive coupling, NFC or other communication technologies operable at ranges less than 10-20 cm) and far-field communication technologies (e.g., radiofrequency (RF) telemetry according to the 802.11 or Bluetooth® specification sets, or other communication technologies operable at ranges greater than near-field communication technologies).

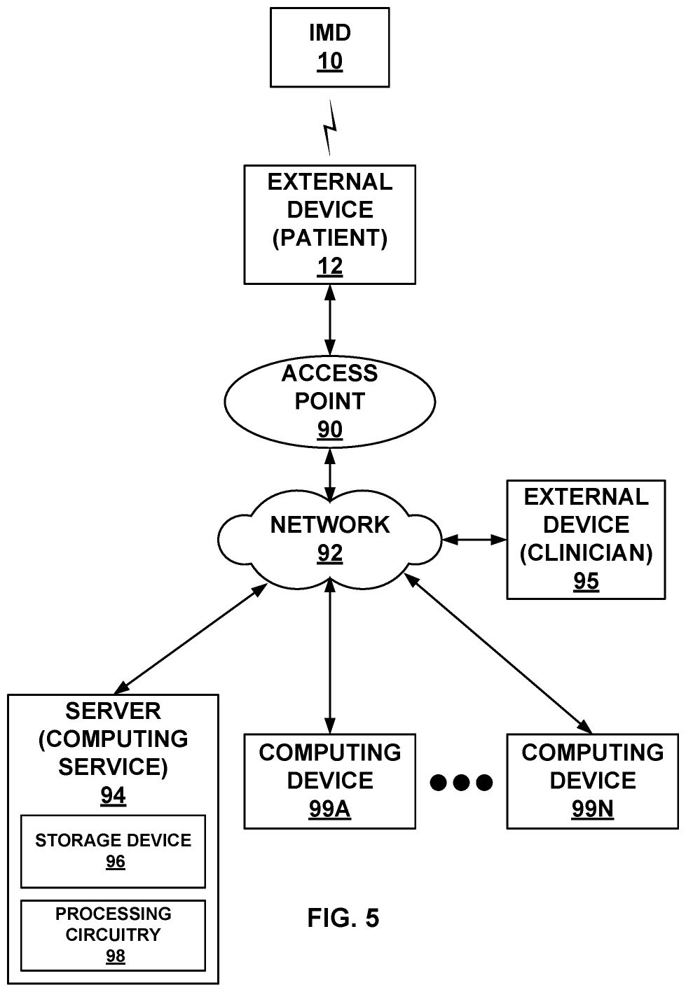

External device 12 may be used to configure operational parameters for IMD 10. External device 12 may be used to retrieve data from IMD 10. The retrieved data may include values of physiological parameters measured by IMD 10, indications of episodes of arrhythmia or other maladies detected by IMD 10, and sensor signals recorded by IMD 10. For example, external device 12 may retrieve cardiac EGM segments recorded by IMD 10 due to IMD 10 determining that an episode of arrhythmia (e.g., asystole) or another malady occurred during the segment. As another example, external device 12 may receive sensor data, metric values, or other data related to the techniques described herein from IMD 10. As will be discussed in greater detail below with respect to FIG. 5, one or more remote computing devices may interact with IMD 10 in a manner similar to external device 12, e.g., to program IMD 10 and/or retrieve data from IMD 10, via a network.

Monitoring service 6 refers to a computing service, which may be similar to that provided by the Medtronic CareLink® Network, that communicates with IMD 10 directly over a network connection and/or indirectly through external device 12. One or more aspects of medical system 2 of FIG. 1 may be implemented with networking infrastructure connecting computing devices of monitoring service 6 with IMD 10 and/or external device 12. As described herein, uploads of various datasets (e.g., health event data, samples of patient data, longitudinal diagnostic information, and/or the like) may trigger monitoring service 6 into adjudicating initial detections by IMD 10 of health events (e.g., cardiac events) and if needed, adjusting IMD 10's device history of IMD 10 to more accurately reflect patient 4's health.

Processing circuitry of medical system 2, e.g., of one or more devices of monitoring service 6, IMD 10, external device 12, and/or of one or more other computing devices, may be configured to perform the techniques described herein. The processing circuitry of medical system 2 may employ various known mechanisms to capture (e.g., samples) of various patient data over a period of time, analyze captured physiological parameter values for indicia of any health events including non-trivial changes in patient health. Then, the processing circuitry of medical system 2 may determine whether to remove the captured patient data for the health events from memory resources and from a medical device history/diagnostic information. In some examples, the processing circuitry of medical system 2 analyzes a cardiac EGM or ECG and other patient activities sensed by IMD 10 and may identify indicia of a cardiac episode, such as an arrhythmia, or another cardiac event that either has occurred or is occurring in patient 4.

The present disclosure envisions medical devices that are equipped with a number of hardware/software components to implement a number of different example techniques. IMD 10, as one example medical device, may be configured with detection logic to implement techniques to determine whether patient 4 is experiencing/has experienced/will (imminently) experience an example cardiac episode. As described in the present disclosure, the detection logic may employ a number of compatible mechanisms to successfully implement the above technique, such as machine learning model and/or a decision tree, where each mechanism prescribes criterion that the detection logic may use for distinguishing patient data indicative of a true cardiac episode from patient data that does include a true cardiac episode. External device 12 and/or one or more devices of monitoring 6 may use logic (e.g., adjudication logic) to verify an initial detection of an arrhythmia episode (or another health event) as a true positive or reject that initial detections as a false positive.

Although described in the context of examples in which IMD 10 that senses the cardiac EGM or ECG, example systems including one or more implantable, wearable, or external devices of any type configured to sense the cardiac EGM or ECG may be configured to implement the techniques of this disclosure.

In some examples of medical system 2, processing circuitry in a wearable device may execute same or similar logic as the logic executed by processing circuitry of IMD 10 and/or other processing circuitry as described herein. In this manner, a wearable device or other device may perform some or all of the techniques described herein in the same manner described herein with respect to IMD 10. In some examples, the wearable device operates with monitoring service 6 to access resources (e.g., computing services) in the same manner described herein with respect to IMD 10 and/or external device 12. In some examples, the wearable device operates with IMD 10 and/or external device 12 as potential providers of computing/storage resources and sensors for monitoring patient activity and other physiological parameters. For example, the wearable device may communicate the patient data to external device 12 for storage in non-volatile memory and for computing values for various parameters including patient physiological parameters or values corresponding to sensor measurements including sensed patient activity.

According to some example techniques, while monitoring patient physiology and detecting any changes in patient health (e.g., health events), medical system 2 of this disclosure may be directed to automatically enroll patient 4 with monitoring service 6, for example, as an authorized user with access privileges to resources (e.g., functionality) provided over a network connection. In addition to patient 4 as an authorized user, medical system 2 may successfully enroll a medical device for patient 4, such as IMD 10, or a software application running on a patient device, such as a medical application running on external device 12. This may be accomplished before or while the processing circuitry of medical system 2 runs logic to analyze the monitored patient activity/detected changes in patient health.

At some point before or at a same time that the medical application is downloaded and/or executed, external device 12 may receive a GUID (e.g., in an identity (ID) token) assigned by monitoring service 6 and/or a third party system. Depending on which security architecture is implemented in monitoring service 6, the instance of the medical application (or a separate process) may execute code to request and in a timely response, receive a GUID (e.g., in an identity (ID) token) issued by monitoring service 6 and/or a third party system. An example token may include attribute information (e.g., claims) about patient 4. The same attribute information may be used to auto-generate a completed enrollment request in at least the following examples.

Responsive to receiving and/or accessing the GUID (e.g., from the identity token), the medical application may be configured to auto-generate an enrollment request using some (of not all) of the GUID and additional information as prescribed by monitoring service 6. In one examples, the medical application may insert information from one or more claims in the identity token into the enrollment request. Hence, the medical application may leverage information provided in a token and/or information available from a local source (e.g., a physically connected source or a communicatively coupled source) to automatically complete the enrollment request. If needed, application 88 may add, modify, and/or remote attributes from any enrollment request.

In some examples, an initial use of a medical device and/or a medical application may be conditioned on successful enrollment with monitoring service 6. In some examples, a software application assumes operational control over IMD 10 upon completion of an enrollment process for patient 4. The software application may be configured to direct operation of IMD 10 with respect to health event monitoring/detection, for example, by programming device settings (e.g., including ML model parameters). The software application may be further configured to provide functionality to support/enhance the health event monitoring/detection by IMD 10. As such, the software application may exchange data with IMD 10 via a native interface. The software application may be a client application for monitoring service 6 and configured to facilitate access to a patient account.

The present disclosure describes medical systems and techniques that leverage a “GUID” (and possibly other information) for the completed enrollment request recognizes the variety of embodiments for “GUID” and its equivalents. An example GUID generally represents a “Globally Unique Identifier” but is not limited with respect to numerical value. An example GUID may be any N-byte (e.g., 16-byte or 128-bit) integer expressed (e.g., on a GUI) in hexadecimal notation (e.g., 32 symbols or digits). An example GUID may be implemented, by software programs, in a system of GUIDs. In a computing device such as external device 12, the software programs may leverage the system of GUIDs in one of several mechanisms (e.g., a data model) for performing a number of data management (e.g., database system) tasks, such as uniquely identifying a physical/virtual memory location of a specific data object amongst set of data objects. Example embodiments in which GUIDs uniquely identify (and in some examples compose part of) one or more data objects include MICROSOFT Windows® Registry entries, relational database keys for uniquely identify each record in database table, a data structure such as an index for retrieval/lookup of a row or rows given values for the columns (fields), and/or the like. When identifying a specific data object e.g., a patient account), the example GUID may be configured with information that will remain constant and unique across time. Some applications sometimes use fields such as full name, email address, and/or a phone number.

The example GUID may include and/or associate with sufficient information for new account registration. This information may be referred to herein as attributes. In general, “attributes” refer to information, for example, to identify the medical application, patient 4, IMD 10, and/or their patient account with monitoring service 6. Monitoring service 6 and/or a third party system may define an identifier, which may be the GUID described herein or another identifier. One example identifier may uniquely identify patient 4 across hardware/software components (e.g., devices and applications) of monitoring service 6. One example identifier may be specific to the patient account of patient 4. One example identifier may be an immutable identifier (e.g., an object ID attribute) as defined by monitoring service 6 and/or the third party system. Some example identifiers may expire, requiring refreshing, while other example identifiers cannot be reused. Some claims may further identify patient 4 by way a last name or subject attribute. Some example identifiers may be pre-authorized by monitoring service 6 and then, transmitted to external device 12 for storage prior to (e.g., in preparation of) installing a copy of the medical application.

In other examples, the medical application may generate an enrollment request with different information except for the GUID. Monitoring service 6 may change its registration process and require additional information for new patient accounts. Even in examples where monitoring service 6 does release data to populate the enrollment request, the medical application may be configured to create any combination of attribute information to complete the enrollment request, for example, given that the GUID may operate as a database key to specifically identify a new account for patient 4 in a database for all patient accounts of monitoring service 6. In one example, the medical application may generate the GUID alongside or instead of an identity/access token. In another example, by prompting monitoring service 6 and/or a third-party system to return an account confirmation number through which the patient may obtain new account authorization, application 88 may include the account confirmation number in a request for enrollment. This GUID may expire and when a new GUID is requested, the new GUID may be different but still uniquely identify patient 4. As an alternative to GUIDs, monitoring service 6 may implement encrypted blobs.

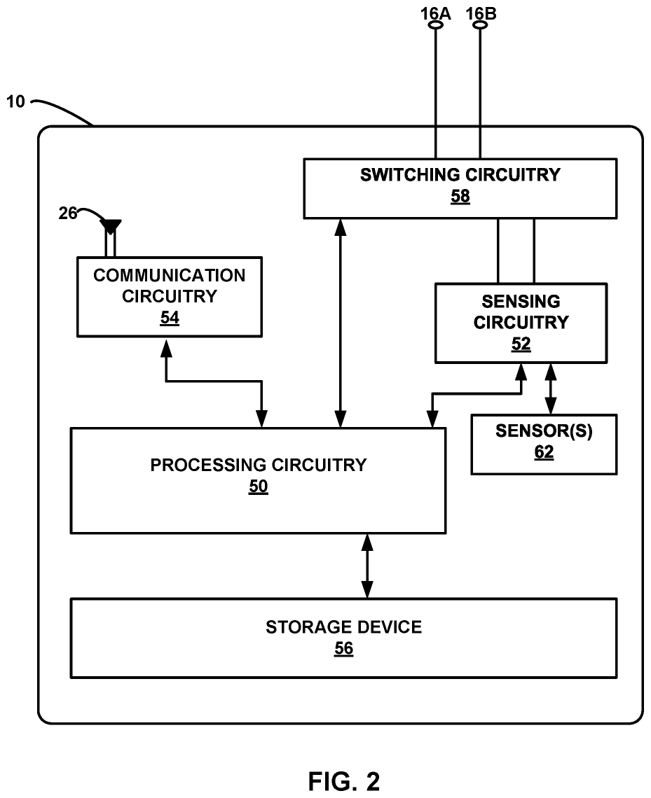

FIG. 2 is a functional block diagram illustrating an example configuration of IMD 10 of FIG. 1 in accordance with one or more techniques described herein. IMD 10 described herein refers to a medical device capable of automating patient registration with a computing service that supports functionality related to patient health monitoring and cardiac event detection.

In the illustrated example, IMD 10 includes electrodes 16A and 16B (collectively “electrodes 16”), antenna 26, processing circuitry 50, sensing circuitry 52, communication circuitry 54, storage device 56, switching circuitry 58, and sensors 62. Although the illustrated example includes two electrodes 16, IMDs including or coupled to more than two electrodes 16 may implement the techniques of this disclosure in some examples.

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be selectively coupled to electrodes 16 via switching circuitry 58, e.g., to sense electrical activity of the heart of patient 4, for example by selecting electrodes 16 and polarity, referred to as the sensing vector, used to sense the cardiac electrical activity, as controlled by processing circuitry 50. Sensing circuitry 52 may sense signals from electrodes 16, e.g., to produce cardiac EGM data or ECG data as examples of the patient data. Sensing circuitry 52 may monitor signals from sensors 62 to process various sensor measurements to include as part of the patient data. Processing circuitry 50 may control one or more of sensors 62 to sense the patient data in some form; examples of one or more sensors 62 to sense patient data include an accelerometer (e.g., a three-axis accelerometer), a pressure sensor, an optical sensor, a gyroscope, a temperature gauge, a moment transducer, and/or the like. Various metrics enable standardized measurement(s) for each sample (e.g., timestamp) of the sensed patient data and differentiation between multiple samples. In some examples, sensing circuitry 52 may include one or more filters and amplifiers for filtering and amplifying signals received from electrodes 16 and/or sensors 62.

Communication circuitry 54 may include any suitable hardware, firmware, software or any combination thereof for communicating with another device, such as external device 12, another networked computing device, or another IMD or sensor. Under the control of processing circuitry 50, communication circuitry 54 may receive downlink telemetry from, as well as send uplink telemetry to external device 12 or another device with the aid of an internal or external antenna, e.g., antenna 26. In addition, processing circuitry 50 may communicate with a networked computing device via an external device (e.g., external device 12) and a computer network, such as the Medtronic CareLink® Network. Antenna 26 and communication circuitry 54 may be configured to transmit and/or receive signals via inductive coupling, electromagnetic coupling, Near Field Communication (NFC), Radio Frequency (RF) communication, Bluetooth, WiFi, or other proprietary or non-proprietary wireless communication schemes.

In some examples, storage device 56 includes computer-readable instructions that, when executed by processing circuitry 50, cause IMD 10 and processing circuitry 50 to perform various functions attributed to IMD 10 and processing circuitry 50 herein. Storage device 56 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Storage device 56 may store, as examples, programmed values for one or more settings and/or operational parameters of IMD 10 and/or data collected by IMD 10 for transmission to another device using communication circuitry 54. Data stored by storage device 56 and transmitted by communication circuitry 54 to one or more other devices may include the patient data described herein for suspected changes in and/or indications of changes in patient health.

Sensing circuitry 52 may capture signals from electrodes 16 and/or any one or more of sensors 62, for example, to produce the patient data for processing by hardware/software (e.g., within IMD 10, external device 12, and/or another device), thereby facilitating (e.g., remote) monitoring and detecting changes in patient health (e.g., by an external computing system such as a network-accessible device running a computing service). The above-mentioned hardware/software may include software applications configured with logic to generate additional patient data, for example, data further describing patient health in terms of a health event history over a time period. In one example, processing circuitry 50, executing logic herein referred to as detection logic, applies various criteria to a timestamped sample of sensor measurements to determine if the sample is evidence of an arrhythmia likely to cause a change in patient health or, otherwise, negatively affect the patient's heart.

Sensing circuitry 52 converts to digital form signals corresponding to the sensed electrical activity and provides the digitized signals to processing circuitry 50 for an initial detection analysis performed by the detection logic. The patient data stores the cardiac EGM data encompassing a period of time (e.g., during which a cardiac episode may have occurred) and, in some examples, includes a sequence of values representing waves/waveforms (e.g., ECG-type waveforms) of a cardiac rhythm. It should be noted that the cardiac EGM data (e.g., for a typical ECG) records a series of samples representing points on waves (e.g., the P wave, Q wave, R wave, S wave, T wave and U wave), intervals (e.g., PR interval, QRS interval (also called QRS duration), QT interval or RR interval), segments (e.g., PR segment, ST segment or TP segment), complex(es) (e.g., QRS complex), and other components. The detection logic may direct processing circuitry 50 to apply a pattern recognition technique to interpret electrical activity vectors recorded in corresponding cardiac EGM data as one or more of the above components. In some examples, the waveform may indicate an initial detection of a cardiac event. Processing circuitry 50 generates for display output data indicative of a particular cardiac event type as a classification of the detected cardiac event.

Processing circuitry 50 and/or sensing circuitry 52 may read/write the patient data from/to storage device 56. Processing circuitry 50 and/or sensing circuitry 52 may cooperate to continuously record (e.g., monitor) the cardiac EGM data or ECG data, for example, as a graph of two-dimensional points and/or vectors. Storage device 56 may store a variety of additional data including information identifying IMD 10 in terms of its type of medical device, classification of algorithm (e.g., machine learning algorithm) implemented as detection logic for various health events, state of device settings currently programmed into the detection logic, other operational characteristics, amongst other examples of identity information representative of IMD 10.

Processing circuitry 50 and/or communication circuitry 54 may upload the patient data via a communication channel (e.g., a BLUETOOTH connection) to a remote device, such as external device 12 of FIG. 1. Examples of the uploaded patient data include health event data, which may be generated by the above-mentioned detection logic configured to detect occurrences of cardiac episodes, for instance, by determining whether the cardiac EGM data or ECG data is indicative of such cardiac episodes. The patient data may further include other data corresponding to the health event history, such as data identifying a patient group/class.

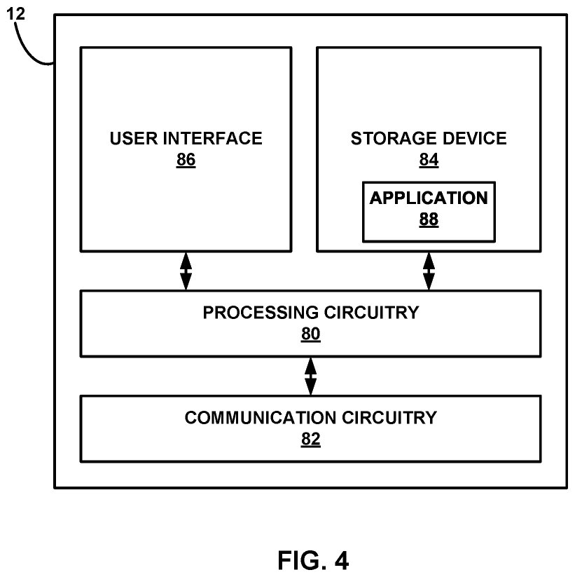

The above-mentioned patient data, generated (in part) by sensing circuitry 52 from the captured sensor signals that encode sensed patient physiology information including the patient's cardiac activity as described herein, serves as input to the detection logic that, while executing in processing circuitry 50, detects one or more samples of patient data indicative of at least one health event including a cardiac episode. The detection logic refers to program code (e.g., processor-executable instructions) for performing the initial detection analysis on the patient data for occurrences of different types of health events. Processing circuitry 50 may generate a health event history indicating points-in-time of probable health events (i.e., positive detections). As described in further detail for FIG. 4, external device 12 is an example of a device configured to operate with IMD 10, for example, by providing access to a software application for extending native capabilities of IMD 10 with respect to monitoring and detecting health events. The software application further provides a network connection for communicating with a computing service such as monitoring service 6 of FIG. 1. One of or both the computing service and the software application running on an external device enhance performance of IMD 10 with additional functionality, such as adjudication functionality configured to identify and/or eliminate any false positive detections of health events in the patient data.

The present disclosure describes IMD 10 as part of a medical system capable of automatically enrolling IMD 10 and its patient, patient 4 of FIG. 1, with the above-mentioned computing service via any one of a number of techniques. In one example, IMD 10, after a successful implant into patient 4, commences operation to monitor patient health responsive only to completion of an enrollment process. In external device 12, registration logic automates at least a portion of the enrollment process, for example, by pre-populating data fields of an enrollment request with required attributes. The present disclosure describes a number of mechanisms through which accurate patient information (including pre-determined credentials) is provided to complete the enrollment request. The present disclosure further describes at least one mechanism to establish new credentials, for instance, by auto-generating identity data for authentication by the computing service.

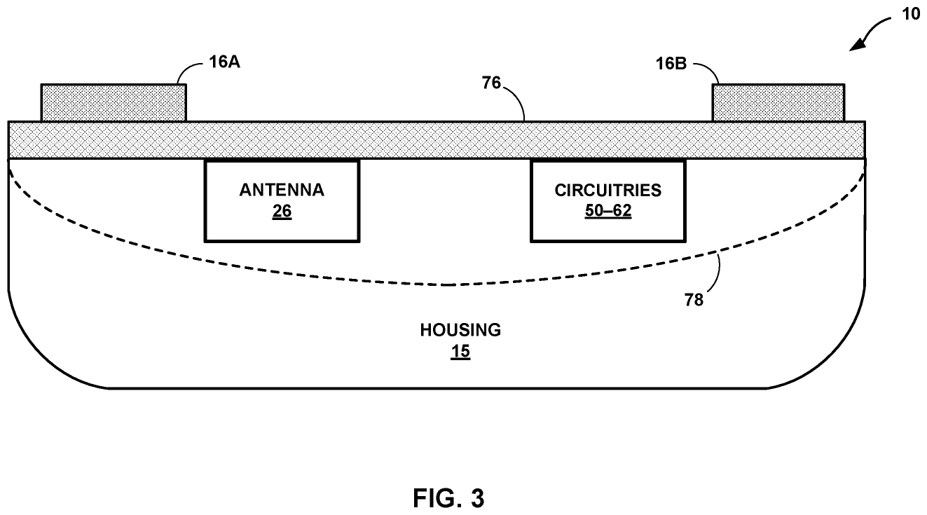

FIG. 3 is a conceptual side-view diagram illustrating an example configuration of IMD 10 of FIGS. 1 and 2. While different examples of IMD 10 may include leads, in the example shown in FIG. 3, IMD 10 may include a leadless, subcutaneously-implantable monitoring device having a housing 15 and an insulative cover 76. Electrode 16A and electrode 16B may be formed or placed on an outer surface of cover 76. Circuitries 50-62, described above with respect to FIG. 2, may be formed or placed on an inner surface of cover 76, or within housing 15. In the illustrated example, antenna 26 is formed or placed on the inner surface of cover 76, but may be formed or placed on the outer surface in some examples. In some examples, insulative cover 76 may be positioned over an open housing 15 such that housing 15 and cover 76 enclose antenna 26 and circuitries 50-62, and protect the antenna and circuitries from fluids such as body fluids.

One or more of antenna 26 or circuitries 50-62 may be formed on the inner side of insulative cover 76, such as by using flip-chip technology. Insulative cover 76 may be flipped onto a housing 15. When flipped and placed onto housing 15, the components of IMD 10 formed on the inner side of insulative cover 76 may be positioned in a gap 78 defined by housing 15. Electrodes 16 may be electrically connected to switching circuitry 58 through one or more vias (not shown) formed through insulative cover 76. Insulative cover 76 may be formed of sapphire (i.e., corundum), glass, parylene, and/or any other suitable insulating material. Housing 15 may be formed from titanium or any other suitable material (e.g., a biocompatible material). Electrodes 16 may be formed from any of stainless steel, titanium, platinum, iridium, or alloys thereof. In addition, electrodes 16 may be coated with a material such as titanium nitride or fractal titanium nitride, although other suitable materials and coatings for such electrodes may be used.

FIG. 4 is a block diagram illustrating an example configuration of components of external device 12. In the example of FIG. 4, external device 12 includes processing circuitry 80, communication circuitry 82, storage device 84, and user interface 86.

Processing circuitry 80 may include one or more processors that are configured to implement functionality and/or process instructions for execution within external device 12. For example, processing circuitry 80 may be capable of processing instructions stored in storage device 84. Processing circuitry 80 may include, for example, microprocessors, DSPs, ASICs, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80.

Communication circuitry 82 may include any suitable hardware, firmware, software or any combination thereof for communicating with another device, such as IMD 10. Under the control of processing circuitry 80, communication circuitry 82 may receive downlink telemetry from, as well as send uplink telemetry to, IMD 10, or another device. Communication circuitry 82 may be configured to transmit or receive signals via inductive coupling, electromagnetic coupling, NFC, RF communication, Bluetooth, WiFi, or other proprietary or non-proprietary wireless communication schemes. Communication circuitry 82 may also be configured to communicate with devices other than IMD 10 via any of a variety of forms of wired and/or wireless communication and/or network protocols.

Storage device 84 may be configured to store information within external device 12 during operation. Storage device 84 may include a computer-readable storage medium or computer-readable storage device. In some examples, storage device 84 includes one or more of a short-term memory or a long-term memory. Storage device 84 may include, for example, RAM, DRAM, SRAM, magnetic discs, optical discs, flash memories, or forms of EPROM or EEPROM. In some examples, storage device 84 is used to store data indicative of instructions for execution by processing circuitry 80. Storage device 84 may be used by software or applications running on external device 12 to temporarily store information during program execution.

Data exchanged between external device 12 and IMD 10 may include operational parameters. External device 12 may transmit data including computer readable instructions which, when implemented by IMD 10, may control IMD 10 to change one or more operational parameters and/or export collected data. For example, processing circuitry 80 may transmit an instruction to IMD 10 which requests IMD 10 to export collected data (e.g., asystole episode data) to external device 12. In turn, external device 12 may receive the collected data from IMD 10 and store the collected data in storage device 84. The data external device 12 receives from IMD 10 may include various patient data including episode data, sensor measurements, sensed electrical activity, and other patient data described herein. Processing circuitry 80 may implement any of the techniques described herein to analyze data from IMD 10 to determine parameter values e.g., to determine whether the patient is experiencing a change in health e.g., based upon one or more criteria.

A user, such as a clinician or patient 4, may interact with external device 12 through user interface 86. User interface 86 includes a display (not shown), such as a liquid crystal display (LCD) or a light emitting diode (LED) display or other type of screen, with which processing circuitry 80 may present information related to IMD 10, e.g., daily metric values, indications of changes in daily metric values, and indications of changes in patient health that correlated to the changes in daily metric values. In addition, user interface 86 may include an input mechanism configured to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 12 and provide input. In other examples, user interface 86 also includes audio circuitry for providing audible notifications, instructions or other sounds to the user, receiving voice commands from the user, or both.

In one example, external device 12 may generate a database to arrange database records) indicating points-in-time of probable health events (i.e., positive detections). The database records may be arranged chronologically (e.g., a health event history). External device 12 is configured to operate with IMD 10, for example, by providing access to a software application for extending local resources and native capabilities of IMD 10 with respect to monitoring and detecting health events. A software application further provides a network connection for communicating with a computing service. One of or both the computing service and the software application enhance performance of IMD 10 with additional functionality, such as adjudication functionality configured to identify and/or eliminate any false positive detections of health events in the patient data.

External device 12 may be configured with to initiate operation of a computing service for a medical device (e.g., post-implantation) by performing, via a computer network, an automatic enrollment process. External device 12 may be a computing device (e.g., a network-accessible computing device such as a mobile phone or smart phone) capable of running the above-mentioned software application as a 0-click or 1-click solution for enrolling the patient and/or the medical device with a computing service. External device 12, which may be known as an edge device or a patient device for IMD 10 of FIGS. 1-2, may be capable of automating patient registration with the computing service that supports functionality related to patient health monitoring and health event detection.

The computing service generally refers to a computing system (e.g., a cloud service) that communicates, via a network protocol/service, to devices such as external device 12. An example computing service may be a web server configured with an Internet connection to a web application whose resources are consumable by external device 12 or another patient device. Software applications, such as medical application 88 of FIG. 4, may utilize communication circuitry 82 to generate a network connection to the computing service. Monitoring service 6 of FIG. 1 is an example of the computing service.

In the example of FIG. 4, medical application 88, or simply application 88, may be an example of the above-mentioned software application for completing the enrollment process and commencing operation of monitoring service 6, for example, enabling functionality of application 88, for example, in support current medical device functionality, to add new medical device functionality and, possibly, commence or calibrate operation of the medical device. In general, application 88 includes logic (e.g., program code such as a script) configured to run functionality on behalf of the above-mentioned computing service for the benefit of the patient. The logic of application 88 may be executable on processing circuitry or embodied on logic circuitry (e.g., system-on-chip (SoC)).

In general, an initial code section of application 88 (or a separate software element as described herein) prepares an initial communication responsive to receiving input comprising a GUID. The present disclosure generally refers to this GUID as a globally unique identifier for a patient having an implanted medical device and, in some examples, the only information required for enrolling the patient into a computing service, such as monitoring service 6. The present disclosure does not imply or represent the GUID as being further limited. It should be noted that the initial communication may include only the GUID and still achieve an automatic enrollment for the patient having the implanted medical device. It should be further noted that the GUID may be configured in a number of different ways and/or for variety of purposes. For example, the GUID may be an attribute of an credential (e.g., an access token) or form part of a network address (e.g., a logical address such as a URL) directed to an instance of a registration process executed by a computing device of monitoring service 6. Another example GUID may a database key configured to retrieve the required information for a successful enrollment.

In any example, having access to an example GUID, application 88 may be configured to access information corresponding to GUID and then, use that information to generate a completed enrollment request. Application 88 may include, in the initial code section, processor-executable instructions configured to communicate the completed enrollment request. Assuming patient 4 or another user is performing a manual enrollment, the processor-executable instructions of application 88 may generate a GUI button that when activated, sends the user to a URL for a device of monitoring service.

Otherwise, the processor-executable instructions of application 88 may automatically generate and then, communicate a request (e.g., a HTTP request or any RESTful request) to the URL directed towards a device of monitoring service 6. This request is similar to any request communicated by activating the GUI button of application 88. One example request may be characterized as a credential request (e.g., an access token request). Another example request may be an authenticated request such as a request having (e.g., as claim information) a credential (e.g., an access token) to submit to the device of monitoring service 6.

The credential may be configured in a number of ways. One example credential may include login credentials (e.g., a username and password). Another example credential may include two-factor authorization data (e.g., login credentials plus a security question/answer). Another example credential may be described as an intermediate credential that represents an authorization grant (e.g., an authorization code) by monitoring service 6 and/or a third-party system. Valid credentials may be obtained in exchanged for the intermediate credential.

Application 88 of FIG. 4 generally includes a logical unit, referred to as registration logic, that when executed, is operative to facilitate patient registration for successfully completing the enrollment process. In some examples, the registration logic may be a component of this software application or may be an independent software program to run in a different memory location.

To illustrate by way of example, during an initial use of the medical device by a patient, external device 12 executes the automatic enrollment process to register a specific device user as the patient (e.g., with the medical device implanted). External device 12 may perform the automatic enrollment process by running a dedicated application for the medical device. Alternatively, the medical device may run the dedicated application to register the patient with the computing service. External device 12 and the medical device are Bluetooth Low Energy (BLE)-enabled or Bluetooth-enabled and therefore, communicate data to each other via a radio access technology (RAT). Although some examples may utilize a data connection with the medical device, registration logic may be configured in some examples where communications of any data with the medical device, let alone data for the enrollment process, are not enabled. Registration logic may be configured to leverage a network connection for completing the enrollment process and by doing so, avoid communicating data with the medical device.

There are a number of embodiments of the registration logic. Some embodiments of registration logic may be a separate software component from the above-mentioned application, application 88, associated with the medical device and dedicated to the above-mentioned computing service, monitoring service 6 of FIG. 1, while other embodiments of registration logic may be included as a component of such a software application. Some embodiments of registration logic may be a component of the medical device itself. The following description refers to registration logic as a component of application 88 and for that reason, is first downloaded and/or installed on external device 12 prior to performing any example enrollment process. When application 88 is completely downloaded via an Internet connection, processing circuitry 80 may execute the registration logic to perform the enrollment process for other components of application 88. It should be noted that in alternative examples, application 88 may be downloaded before or after registration logic successfully enrolls the patient. In one alternative example, registration logic may be downloaded as an installer program (e.g., in a scripting language) via an Internet connection and when completely downloaded, automatically executed to perform the enrollment process for the patient prior to or after installing application 88.

The present disclosure describes a number of examples of triggering an example enrollment process for registration logic to perform. In general, registration logic may be triggered by receiving certain input. Registration logic may process an example trigger in the form of a proper invocation as described herein. There are a number of suitable mechanisms for any invocation described herein, details of which are provided further below; a description for example steps the automatic enrollment process are provided in the following.

In one example, processing circuitry 80 may execute registration logic to perform steps of an example automatic enrollment process as follows. At an initial or first step, a hardware/software component (e.g., an operating system) of external device 12 may direct processing circuitry 80 to execute code of registration logic, proceeding to a next or second step of the automatic enrollment process. As described herein, a number of hardware/software components may initiate registration of the patient and/or application 88 with monitoring service 6 of FIG. 1. Consider an example where processing circuitry 50 launches a (e.g., native or third-party) software application for IMD 10 (i.e., “a medical application”) and by doing so, triggers the automatic enrollment process for that application, for example, by submitting a new account registration. This may occur in a number of ways such as in response to a user download of application 88 from a remote source (e.g., via a mobile device property known as an “Application Store” or simply “App Store”).

The registration logic running on external device 12 may submit, in an example new account registration, sufficient information for monitoring service 6 to establish a new patient account as described herein. Monitoring service 6 may provide registration logic with a unique identifier in a single attribute (e.g., a Globally Unique Identifier (GUID). The GUID may be generated (e.g., automatically and/or as directed by the patient) prior to or during a post-implant enrollment process as described herein. The present disclosure envisions several attribute types in a variety of embodiments, each defining “sufficient information” for setting up new account with variable requirements. It is possible for monitoring service 6 to use a single attribute, such as a unique medical device identifier or patient last name, for each new account as well as multiple attributes (e.g., patient first, middle, and last names, patient device name, and/or the like).

It also is possible for monitoring service 6 of FIG. 1 to minimize the data submission required from patient 4 for setting up their new account to a set of attributes that can be automatically retrieved by the registration logic, without any user input. Hence, the registration logic may automate medical application/patient account registration upon launch of the registration logic, application 88, or both the registration logic and application 88. In one example, the registration logic may be programmed to include, in a new account request, an identifier that has been designated for application 88 and, without any user involvement, stored (e.g., seeded) in memory. Registration logic and application 88 may be pre-loaded with the above identifier. In another example, the registration logic may be programmed to include, in a new account registration, a user identity for the patient that can be retrieved from a number of sources (e.g., on external device 12 or another patient device) for patient information. In yet another example, monitoring service 6 may not require any identifying information (e.g., attribute information including account/personal credentials).

In some examples where the new account registration involves any degree of user interaction, the registration logic may prepare the new account registration with appropriate attribute information using various data provided by the patient and/or already available in storage device 84. In one example, application 88 and/or the registration logic may generate, for presentation on a computer display, a user interface (UI) such as a GUI on a mobile device screen, which may be followed by the registration logic incorporating, into the new account registration, at least some information that patient 4 entered into a GUI component. Those of ordinary skill should recognize a single page log-in screen as an embodiment of the GUI on the mobile device screen; moreover, the single page log-in screen is compatible with other example UIs, such as a GUI for a desktop device screen.

In some examples, the new account registration may be completed as a condition for successful enrollment. To illustrate by way of example, the registration logic may submit a request for a GUID using a variety of available mechanisms, such as a new account registration. In one example, the new account registration may include various attribute information to identify patient 4, application 88, and/or an implanted medical device (e.g., for IMD 10). For instance, the unique identifier may identify the medical device such as a serial number. The unique identifier may identify application 88 such as an account or object ID. The unique identifier may identify patient 4, for instance, by patient last name. Any one of these unique identifiers may be submitted in a HTTP request configured to receive, as a response, a GUID for completing an enrollment request. The HTTP request may include any required attribute types for the new account registration (e.g., account credentials). The HTTP request may include a token as described herein and directed towards an instance of a complementary registration process running in monitoring service 6. In return, monitoring service 6 communicates an encapsulated GUID or a GUID representing the above two attribute types. The GUID may be predetermined prior to medical device enrollment or generated at the time of enrollment.

Depending on the programming of application 88, any given GUID may enable any number of improvements to the benefit of at least the patient. In one example, application 88 detects and then, extracts the patient's last name from the GUID and performs a communication session (e.g., an encrypted call) into monitoring service 6. The GUID is combined with the patient last name (or an alternative attribute type) to allow monitoring service 6 to release data for pre-populating text fields in registration page presented on the GUI. Monitoring service 6 may validate the patient last name and then, confirm that the user of application 88 (as a medical application) associated with IMD 10 and/or the user of external device 12 (as a patient device) is actually the patient having an implanted medical device.

In one example, launching application 88 as described herein for IMD 10 may cause processing circuitry 80 to execute the registration logic for enabling (e.g., executing) functionality of application 88, which generates content for the GUI and then, opens a network connection with monitoring service 6 to facilitate the automatic enrollment process. Via the GUI presented on user interface 86 external device 12 and/or the network connection to monitoring service 6, the registration logic may package and then, transmit various information (e.g., an identity and other credentials for IMD 10) for enrolling patient 4, application 88, and/or IMD 10 with monitoring service 6. The registration logic may automate an authentication sequence/method to first register (e.g., an account for) application 88 and then, authorize operation of application 88 (e.g., by submitting credentials to successfully login to that account).

Registration, as part of any enrollment process with a complementary computing service, of a hardware device and/or a software application for a patient may enable communications of various data over a network connection to one or more devices of monitoring service 6. In the above automatic enrollment process, external device 12 or another equivalent patient device may be used for registering patient 4 and/or application 88 for IMD 10 and/or only IMD 10, for example, by communicating various data corresponding to IMD 10 in compliance with registration requirements set forth under monitoring service 6. Completing the new account registration in one operation or multiple operations (e.g., in a sequence) as part of the enrollment process, the registration logic may communicate, in one or more datasets, attributes to fulfil the registration requirements as instructed by monitoring service 6. In turn, monitoring service 6 may create a data structure to store, in computer memory, some of the communicated attributes, thereby registering IMD 10 as a medical device for the patient. In addition to establishing a dedicated medical device for the patient, monitoring service 6 may allocate computing resource capacities/capabilities to enhance IMD 10 operation by supporting current functionality and possibly, adding new functionality.

Successful registration of application 88 may require authenticating patient 4 and/or IMD 10. Monitoring service 6 may receive the one or more datasets from external device 12 and proceed to set up an account for patient 4. In one example, monitoring service 6 may combine various attributes into a unique dataset for identifying communications from application 88 and in some instances, which patient device (e.g., external device 12) is running application 88. It should be noted that any unique grouping of attributes can form a deterministic (database) relation with a specific account for patient 4 and their medical device, IMD 10; in some examples, one or more attributes may describe application 88 while other attributes may describe patient 4 and/or IMD 10.

In general, monitoring service 6 configures a database with data tables having the GUID described herein as an index, thereby allowing an immediate retrieval of account data for specific patient. Monitoring service 6 can further arrange a single data table with respective entries of various attributes, including any information used for the account set up. In this manner, the data table is configured to map a specific account's GUID to data corresponding to the specific patient, a specific medical device, and/or a specific medical application. Leveraging such a database configuration, monitoring service 6 may distribute GUIDs to patients (e.g., patients in general or patients of a same medical device) and initiate their enrollment with a computing service that provides the patients with enhanced medical care and other benefits.

Each computing service runs on one or more computing device of monitoring service 6, which manages and controls access to a number of computing services as described herein. At least one service can benefit a particular patient; for example, based on various patient data for a particular patient (e.g., patient 4), the computing service may perform functionality related to diagnostic and health event monitoring. A medical device of the particular patient may store at least some of the patient data relied upon by the computing service and therefore, to ensure accuracy of medical device operation, monitoring service 6 (via a different computing service) may perform device maintenance and management functionality. In general, the computing services enables functionality designed to benefit (e.g., and on behalf of) the particular patient with enhanced medical device, for example, by enabling production of accurate patient data, immediate diagnosis of patient medical conditions, continuous monitoring of patient physiology and detection any heath events corresponding to that patient physiology, and/or the like.

In some examples, the distribution of GUIDS may result in almost immediate patient account creation and contemporaneous enrollment for one or more of the above computing services of monitoring service 6. The patient may upload or otherwise transmit their individual GUID through a variety of mechanisms; as a response to receiving the individual GUID, monitoring service 6 may extract from a single data table an entry having required account setup information for that patient and proceed to establishing a new patient account. As described herein, successful enrollment further should benefit the patient by commencing medical device operation and then, supporting its health event monitoring and detection functionality.

Application 88 may establish a communication channel (e.g., uplink telemetry) with the medical device for receiving/sending data and generate a GUI for presentation on a patient device for entering/modifying/removing various data. Using the communication channel, application 88 may record and collect medical device data. The communication channel may not be necessary for account creation in some examples.

Using the GUI, the patient may enter the GUID for application 88 to facilitate patient account creation and commence medical device operation in a number of ways. In one example, application 88 may use the GUID in a submission of a conventional request for a new account and/or service enrollment. Generally, a new account request for any patient identifies specific patient data corresponding to a number of attributes in satisfaction of one or more requirements for the new account. While the conventional request may require user input via the GUI, a request in accordance with the present disclosure may be auto-generated to include the requisite attribute information and then, prepared for the submission without any human interaction (e.g., auto-submitted).

While the above describes examples where a new account request is substantially autonomous, other examples have at least some human involvement. Instead of physically entering the above-mentioned attributes, patient 4 may input, via the GUI, the GUID for application 88 to leverage in generating a complete request. Application 88 may use the GUID to retrieve (e.g., from an external source) the attributes for satisfying the requirements for a new account and completing the request. In response to the retrieval, application 88 may automatically generate the new account request as described herein. In some examples, the conventional request may be in form of a computerized document (e.g., comprised of HTML or XML data) configured to identify the retrieved attributes. Transmission of the computerized document, by external device 12, to a device of monitoring service 6 may prompt creation of the new account.

One example computerized document, which may be known as a web or Internet document, may delineate the required patient data for submission in as a new account request. An appropriate server of monitoring service 6 may generate the example computerized document for presentation to the patient on a display of a patient device via the GUI of application 88 or a GUI of a browser application. The server may implement functionality for enabling the above submission to monitoring service 6.

An electronic form is one embodiment of the example computerized document where form fields itemize different attributes corresponding to the patient behind the new account request. Instead of having a user enter input of respective attributes into the form fields, application 88 may pre-populate the form fields with the correct attribute information into a request in accordance with the present disclosure. By doing so, application 88 avoids any delay in enrolling the patient with monitoring service 6.

As described herein, the appropriate server of monitoring service 6 may communicate at least a portion of the correct attribute information for application 88 to pre-populate the electronic form requesting a new account for the patient or, alternatively, provide application 88 with the pre-populated electronic form. In addition, from one or more of a variety of sources, application 88 may have readily available the correct attribute information to pre-populate at least a portion of the electronic form. At any time prior to implantation of the medical device, one or more attributes of specific patient data may be pre-determined and stored in preparation for the implantation. Responsive to the medical device implantation, application 88 may retrieve the pre-determined attribute(s) and/or any attribute information that application 88 determines from various data stored in external device 12 and/or received from the monitoring service 6 and then, generate the pre-populated electronic form. In this manner, application 88 may transmit, with little or no involvement from the patient, the correct attribute information for the new account to the appropriate server of monitoring service 6.

In some examples, the GUID may include a dataset of specific patient data; application 88 may use the GUID to determine some (if not all) the correct attribute information to pre-populate the electronic form. In one example, the GUID may be comprised of a combination of two or more of such attributes for application 88 to parse and then, extract as separate attributes. The GUID may be configured as an encapsulated GUID or a GUID representing one or more data attributes. The GUID may operate as its own attribute, for example, as a medical device identifier for IMD 10 of FIG. 1. The GUID may also reference one or more attributes used in the enrollment (e.g., in a mapping), such as when operating as an index of an attribute data table, each table entry functions as an example mapping between the GUID and a dataset comprises of various attributes. The dataset is unique to patient 4 amongst a patient population and therefore, the GUID is deterministic.

Application 88 may delineate the correct attribute information for the new account in a message for transmission by external device 12 to the appropriate server of monitoring service 6. In one example, the message may be transmitted (e.g., via network communication protocols) as packetized data where each packet's payload comprises a part of a computerized document (e.g., the electronic form described herein) for the new account request. Alternatively, external device 12 sends a message that includes, as a payload, an enumeration of the requisite attributes being used to set up the new account, for example, without pre-populating any electronic document. In one alternative example, the message is composed for transmission to the appropriate server of monitoring service 6 in response to the implantation of the medical device. As an option for any request, application 88 may combine the GUID with the enumerated attributes, for example, in a message or in the computerized document. As another alternative, application 88 may submit only include the GUID in a new account request. Monitoring service 6 may retrieve, from incoming message(s), new account information comprising attributes (possibly including the GUID) for the patient, application 88, the medical device, and/or the like.

To illustrate by way of example where IMD 10, a new implanted medical device for patient 4 of FIG. 1, corresponds to a GUID comprised of at least one attribute for validating IMD 10 and/or application 88, external device 12 may communicate a GUID request to receive that GUID. Application 88, a medical application to support IMD 10, or another application (e.g., a browser application) may submit the GUID request in a variety of ways. In response to the GUID request, the appropriate server of monitoring service 6 may generate the GUID to return to external device 12 or another patient device for successfully enrolling patient 4 into monitoring service 6.

Application 88 may be associated with a separate module of specific program code (e.g., registration logic) configured to submit the GUID request and then, generate the computerized document representing the completed enrollment request. In response, Monitoring service 6, in response, may return the GUID to the registration logic, which completes the enrollment request. The registration logic may submit the enrollment request in order to activate application 88 and initiate execution of its functionality.

Alternatively, the registration logic may generate a GUID and then, confirm that GUID is unique from monitoring service 6. From any number of sources, registration logic of application 88 may access requisite attribute information to satisfy restrictions set forth by monitoring service 6, for example, restrictions concerning new patient account creation and then, generate the GUID for transmission to the appropriate server of monitoring service 6. These restrictions are configured to verify patient 4 as a new implant patient while securing patient data and maintaining patient privacy.

One example restriction set forth under monitoring service 6 may require a submission of certain authentic credentials prior to complementing an enrollment for patient 4. Some of the above attribute information for patient 4 may qualify as credential information. One or more credentials may be used to create a new patient account. The GUID may be known to represent patient 4, for instance, referencing a last name of patient 4 or comprising a last name of patient 4. Registration logic may extract the last name for inclusion in an enrollment request (e.g., where a mapping associated the last name with the GUID); in turn, monitoring service 6 may reference the above-mentioned attribute data table to validate the last name as an authentic identity (i.e., credential) of patient 4. After verifying the last name as an authentic credential for patient 4, monitoring service 6 may proceed to complete a setup process for a new patient account.

Monitoring service 6 may automatically generate a computerized document known as an electronic form with pre-populated new patient account information in its form fields (e.g., text fields). This may be performed instead of application 88 or the above-mentioned registration logic. Monitoring service 6 may communicate with external device 12 to present the pre-populated electronic form on a GUI of application 88 or another application (e.g., the browser application). Monitoring service 6 may reconfigure form fields on the pre-populated electronic form to display data but not to accept that data as input, thereby pre-populating the reconfigured form fields with appropriate attributes of patient 4. Monitoring service 6 may release (e.g., hidden or locked) form fields on the electronic form to pre-populate those released form fields with new account details and/or credentials and other attributes for patient 4.

Monitoring service 6 and external device 12 may cooperate to auto-generate the electronic form with necessary information satisfying the restrictions set forth for enrollment. Submission of the auto-generated electronic form, by external device 12, completes the new account setup (e.g., upon receiving authorization for such a submission from patient 4). As a result of such cooperating, patient 4 and IMD 10 can be successfully enrolled without any human intervention or activity.

External device 12 (via application 88 or the registration logic) may leverage encryption technology for securing the submission of the last name in the enrollment request. In one example, application 88 may initiate an encrypted communication session (e.g., call) with monitoring service 6. Via the encrypted communication session, application 88 may combine the GUID with the last name of patient 4 in an encrypted request for a new account. The last name of patient 4 operates as a credential that monitoring service 6 may authenticate, thereby validating a human user of external device 12 as patient 4 and a recent recipient of IMD 10. Receiving the encrypted request (message) prompts monitoring service 6 into releasing pre-populated text fields of an electronic form being presented on a computer display of external device 12. The release can be effectuated by way of an encrypted response to the external device 12.

Given that an implanted medical device is associated with only one patient and their patient account, monitoring service 6 may establish a unique medical device identifier as an index for a database table of patient accounts. A GUID, such as the above-mentioned encapsulated GUID, is one example of the unique medical device identifier. There may be an example where the unique medical device identifier is a separate attribute from any GUID as described herein, such as when a GUID is configured to identify the patient, a medical application for the medical device, and/or a client application for monitoring service 6.

The unique identifier (e.g., a serial number) may map to a set of credentials for the corresponding patient account. Using this unique identifier and one or more account credentials, monitoring service 6 can verify incoming requests purporting to submit matching account credential information to obtain account access. For this reason, registration logic may establish a new patient account with monitoring service by submitting, in a new account request, a dataset (e.g., a database record) including a unique identifier for IMD 10 and one or more credentials (e.g., username/password, passcode/PIN, verification questions, and/or the like). The dataset may include additional attribute information for patient 4, external device 12, and/or application 88 running on external device 12 or another patient device.

The above-mentioned new account request may be segmented into multiple submissions. In one example, an example sequence (e.g., an authentication/login sequence) may include at least two successive operations where each operation may provide specific attribute data in order to successfully register a medical hardware device and/or a medical software application for a patient with monitoring service 6. For an example registration of IMD 10, application 88 may submit an identity of IMD 10 (and possibly, patient information) for association with a specific patient. It should be noted that the example registration of IMD 10 is, in general, applicable to any medical device. Monitoring service 6 may store a dataset with attributes identifying patient 4 and IMD 10, post-implant, in memory as structured data (e.g., a database record or a data table).

Proper registration confers patient 4 with authorization to use application 88 and/or IMD 10 as a medical device. A successfully registered account enables application 88 to support/enhance operation(s) of IMD 10. Monitoring service 6 may provide a successful enrollment response storing a directive for application 88 to support/enhance operation(s) of IMD 10. In general, application 88 may provide access to functionality for controlling/supporting operation of IMD 10 and/or another medical device (e.g., a wearable device). A clinician for a patient may use monitoring service 6 to review current device setting. Via monitoring service 6, the clinician may submit revised device settings for application 88 to use in programming IMD 10. In this manner, the patient receives better medical care by calibrating IMD 10 operation to the patient's physiology. In some examples where default settings for IMD 10 may not be effective for the patient, application 88 may perform a setup procedure to implement appropriate settings and thus, may be necessary for (e.g., initial) IMD 10 operation.

There are examples where monitoring service 6 does not use a GUID for enrolling patients, for instance, by way of a database of patient attribute datasets indexed by the GUID; instead, some examples implement an alternative enrollment mechanism. In each mechanism and similar to GUID-based implementation, monitoring service 6 enforces restrictions on access from other device due to sensitivity of patient data. In order to overcome these restrictions, monitoring service 6 may prescribe specific conditions for each patient's enrollment request to comply. In one example, the enrollment request must include specific attributes of patient data selected from a database of patient attribute datasets, which monitoring service 6 may use to verify a sender of the enrollment request as patient 4. There may be other criteria to satisfy for enrollment.

One example alternative enrollment mechanism may be implemented in a medical device of a patient, such as IMD 10 of patient 6 where IMD 10 may open a network connection to monitoring service 6 (e.g., over the Internet) and communicate, in an encrypted packet transmission over the network connection, one or more attributes (e.g., a credential) of specific patient data as required by monitoring service 6 to satisfy a verification criterion for enrollment.

Another example alternative enrollment mechanism may facilitate user input of one or more attributes describing a patient, for example, by presenting a GUI and directing the patient or another user to enter a set of credentials for a patient account. A natural user interface (NUI) input device may enable the patient or another user manually entering the specific patient data for authentication. In an encrypted communication session with monitoring service 6, external device 12 may transmit the necessary attribute information to complete the enrollment. One example item of the necessary attribute information includes a credential for an identity of patient 4, such as a password. Another example item of the necessary attribute information includes an answer to a security question. On the GUI presented to patient 4, external device 12 may facilitate a selection of the security question from a drop-down GUI component and then, as part of completing the enrollment, submit both the selection of the security question and the security answer in response to the selection. This item of attribute information provides a number of benefits, for example, by enabling password recovery in an event patient 4 losses the credential.

The example alternative enrollment mechanism may use the GUI to prompt the patient to establish a password (e.g., a self-selected password or a pre-generated/random password). In another example, the password may be combined with other log-in data to establish a multi-part (e.g., two-part) identity to limit access to monitoring service 6. An example two-part identity may include the password and another attribute (e.g., last name or a username). An example multi-part identity may combine the two-part identity with a credential (e.g., verifiable information for an identity of the patient such as Social Security Number (SSN), Personal Identification Number (PIN), Drivers License Number, and/or the like).

One example option after a successful enrollment, external device 12 may generate an authentication token and a refresh token to keep alive the (e.g., encrypted) communication session with monitoring service. In one example, upon completing an initial log-in a patient account, registration logic may configure the authentication token and the refresh token maintain the initial log-in in an active state. The authentication token may expire after a number of months (e.g., 3 months) while the refresh token may be effective for up to one calendar year from an enrollment date. The authentication token and the refresh token may be renewed periodically, for example, through software updates or pushed content from monitoring service 6.

FIG. 5 is a block diagram illustrating an example system that includes an access point 90, a network 92, external computing devices, such as external device 12 for a patient (which is referred to in FIG. 5 as a patient device 12), a server 94 for a computing service, external device 95 for a clinician (which is referred to in FIG. 5 as a clinician device 95), and one or more other computing devices 99A-99N (collectively, “computing devices 99”), which may be coupled to the external computing devices via network 92, in accordance with one or more techniques described herein. In this example, IMD 10 may use communication circuitry 54 to communicate with patient device 12 via a first wireless connection, and to communicate with an access point 90 via a conduit/intermediary such as a medical application running on patient device 12. In the example of FIG. 5, access point 90, patient device 12, server 94, clinician device 95, and computing devices 99 are interconnected and may communicate with each other through network 92.

Access point 90 may include a device that connects to network 92 via any of a variety of connections, such as telephone dial-up, digital subscriber line (DSL), or cable modem connections. In other examples, access point 90 may be coupled to network 92 through different forms of connections, including wired or wireless connections. In some examples, access point 90 may be a user device, such as a tablet or smartphone, that may be co-located with a patient such as patient 4 of FIG. 1. IMD 10 may be configured to transmit data including the patient data described herein, to access point 90. Transmissions of data may be configured in datasets in which attributes describing patient activity and/or device operation over time include: Indications of sensor measurements and other sensed information corresponding to patient activities; indications of errors, alerts/alarms, and performance issues in general; indications of parameter values used in operational settings and/or algorithms implemented in logic executable by processing circuitry 98 and/or indications of changes in patient health. Access point 90 may then communicate the retrieved data to server 94 via network 92.

In some cases, server 94 may be configured to provide a secure storage site for data that has been collected from IMD 10 and/or external device 12. In some cases, server 94 may assemble data in web pages or other documents for viewing by trained professionals, such as clinicians, via clinician device 95 and/or computing devices 99. One or more aspects of the illustrated system of FIG. 5 may be implemented with general network technology and functionality, which may be similar to that provided by the Medtronic CareLink® Network.

In the example illustrated by FIG. 5, server 94 includes a storage device 96, e.g., to store data retrieved from IMD 10, and processing circuitry 98. Although not illustrated in FIG. 5 computing devices 99 may similarly include a storage device and processing circuitry. Storage device 96 may include a computer-readable storage medium or computer-readable storage device. In some examples, storage device 96 includes one or more of a short-term memory or a long-term memory. Storage device 96 may include, for example, RAM, DRAM, SRAM, magnetic discs, optical discs, flash memories, or forms of EPROM or EEPROM. In some examples, storage device 96 is used to store data indicative of instructions for execution by processing circuitry 98.

Processing circuitry 98 may include one or more processors that are configured to implement functionality and/or process instructions for execution within server 94. For example, processing circuitry 98 may be capable of processing instructions stored in storage device 96. Processing circuitry 98 may include, for example, microprocessors, DSPs, ASICs, FPGAs, or equivalent discrete or integrated logic circuitry, or a combination of any of the foregoing devices or circuitry. Accordingly, processing circuitry 98 may include any suitable structure, whether in hardware, software, firmware, or any combination thereof, to perform the functions ascribed herein to processing circuitry 98. Processing circuitry 98 of server 94 and/or the processing circuitry of computing devices 99 may implement any of the techniques described herein to manage data received from IMD 10 and analyze that data for certain information, e.g., to determine whether the health status of patient 4 has changed.

In some examples, one or more of computing devices 99 may be a tablet or other smart device located with a clinician, by which the clinician may program, receive alerts from, and/or interrogate IMD 10. For example, the clinician may access data, including the patient data described herein, collected by IMD 10 through a computing device 99, such as when patient 4 is in in between clinician visits, to check on a status of a medical condition. In some examples, the clinician may enter instructions for a medical intervention for patient 4 into an application executed by clinician device 95 and/or one or more computing devices 99, such as based on a status of a patient condition determined by IMD 10, (e.g., a medical application associated with IMD 10 and running on) patient device 12, server 94, or any combination thereof, or based on other patient data known to the clinician.

Computing device 99A, for example, may transmit the instructions for medical intervention to one or more of the external computing devices located with patient 4 of FIG. 1 or a caregiver of patient 4. For example, such instructions for medical intervention may include an instruction to change a drug dosage, timing, or selection, to schedule a visit with the clinician, or to seek medical attention. In further examples, a computing device 99 may generate an alert to patient 4 based on a status of a medical condition of patient 4, which may enable patient 4 proactively to seek medical attention prior to receiving instructions for a medical intervention. In this manner, patient 4 may be empowered to take action, as needed, to address his or her medical status, which may help improve clinical outcomes for patient 4.

As described herein, monitoring service 6 refers to a number of systems (e.g., a medical system) of some are internal, some are external, and some are a combination. One example system may be a security platform that employs a number of external third-party systems. One example external third-party system may be an identity system for creating, issuing, verifying, revoking, and/or proving credentials. Monitoring service 6 may continue to store patient data internally in a database system in which mappings associate individual patients with various patient information including (e.g., necessary) attribute information for a completed enrollment request. Such an association may be memorialized by monitoring service 6 storing a respective GUID for patient 4 in a corresponding mapping for that patient (e.g., and their patient account). As an alternative, monitoring service 6 may store, in the corresponding mapping for that patient, other attributes (e.g., instead of the respective GUID) including other unique identifiers as described herein (e.g., as an equivalent to the GUID). In either examples, the GUID and/or the other attributes may be used by monitoring service 6 to identify an enrollment request from an instance of application 88.

Some Internet-based examples (e.g., web or mobile applications) of the above-mentioned security platform enable a computing service such as monitoring service 6 to authenticate patient account access and registration requests (e.g., User Account and Authentication Service (UAA)). One example service running on one or more computing devices (e.g., OAuth2™/OpenID™ Connect (OIDC) server) may be configured for centralized identity management through which patient accounts are secured; access/registration is permitted for authorized users. Requests directed to this computing service may be transferred via any suitable communication protocols including secured protocols (e.g., proxied authentication supports standard protocols such as SAML, LDAP and OIDC). The computing service enables web or mobile applications, such as application 88, to process a single sign-on authentication sequence and delegated authorization to the centralized identity management. In these examples, application 88 is configured as web application (client) configured to generate a login/approval UI for the patient or another user and provide user account management via APIs for communicating requests to the computing service.

Using components of FIGS. 1-4 to illustrate, when monitoring service 6 receives the enrollment request and extracts various attributes (e.g., patient identity such as a last name), software libraries and helper functions forming the security platform may use the patient identity information to complete the registration process. Monitoring service 6 performs patient data integration from those application 88 instances. Monitoring service 6 may secure the instances of application 88 by combining the instance on a local patient device and a (cloud or) global instance of the patient account (including any application micro-service(s)) as a client. In other examples, application 88 may run as a typical client application for monitoring service 6. Monitoring service 6 may be configured to generate credentials that application 88 may use to authenticate API function calls (e.g., embedded in the above-mentioned HTTP requests) to application services (e.g., a new account registration process or a security service such as an authentication sequence) operated by monitoring service 6 and/or a third-party system. Monitoring service 6 runs application services that pulls data from an instance of application 88 and/or sources regarding patient 4. Monitoring service 6 may authorize an instance of application 88 to run and perform its native functionality, for example, by issuing new tokens or refreshing expired tokens. Such authorization may be obtained by successfully logging into the patient account. In other examples, external device 12 may authorize execution and operation of instances of application 88 after the initial communication and successful enrollment (with a newly registered account).

In one example, monitoring service 6 may use the GUID (or equivalent attribute(s)) to retrieve various patient information from the database system for setting up a new account for patient 4. In some examples, monitoring service 6 generates a new instance of a patient account for patient 4. In other examples, the registration process may involve an authentication sequence where patient 4 provides a verifiable credential and, in turn, the device of monitoring service 6 returns a successful enrollment response to complete the authentication sequence with application 88.

The present disclosure describes this GUID in a variety of forms, depending on the technology employed by monitoring service 6 (and any authorized third-party systems), and in a substantial number of examples. It should be noted that there are an uncountable number of examples and alternatives for the GUID described herein; the present disclosure envisions a number of additional and/or possible embodiments that one would apply to automatically enroll a patient having an implanted medical device with monitoring service 6.

It should be recognized that a GUID is one example attribute used by such security platforms to identify a respective registration process running on a device of monitoring service 6. GUIDs may be used in tokens implemented by such systems under authority of monitoring service 6. Some tokens (e.g., identity tokes) enable the user to verify to monitoring service 6 that they are the patient they purport (e.g., claim) to be. Some tokens include one or more GUIDs that store/map to sufficient information for a successful enrollment of application 88 and/or patient 4 and their implanted medical device (e.g., IMD 10).

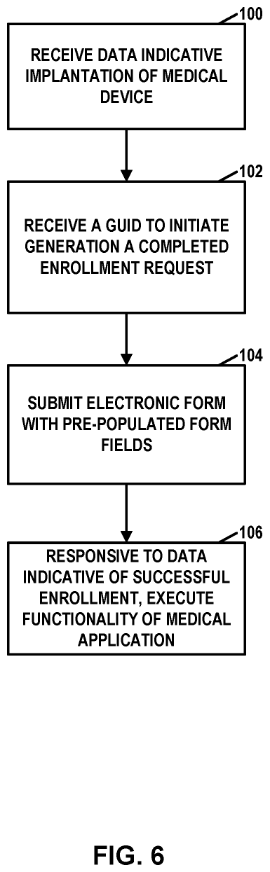

FIG. 6 is a flow diagram illustrating an example operation for automatically enrolling a patient of a medical device with a computing service, in accordance with one or more examples of the present disclosure.

According to the illustrated example of FIG. 6, processing circuitry 80 of external device 12 receives data indicative of an implantation of a medical device (100). Upon having the medical device implanted within their body, a patient may operate a computing device (e.g., a patient device), for example, to generate a completed enrollment request for transmission to a computing service. In the examples described by the present disclosure, the degree of patient involvement varies, for instance, occupying a spectrum of operating modes for the enrollment process: A first mode requiring no patient involvement (e.g., automated upon implantation); a second mode requiring trivial patient involvement (e.g., a minor patient action); a third mode requiring some patient involvement (e.g., some patient involvement by way of a medical application), among others.

The present disclosure describes a variety of techniques where processing circuitry 80 of external device 12 receives an identifier (e.g., a globally-unique identifier or GUID), as input, for initiating (e.g., an automatic) enrollment of patient 4 of FIG. 1 and their implanted medical device, IMD 10. For patient 4, the computing service, monitoring service 6, stores a mapping associating the identifier to at least one dataset of attribute information. As described in details further below, processing circuitry 80 of external device 12 may use a GUID to retrieve one or more attributes of the above mapping and supply sufficient enrollment data to an otherwise incomplete enrollment request, thereby completing that incomplete request and generating a completed enrollment request.

In this manner, patient 4 may benefit from having their personal device, external device 12, configured to detect the implantation of IMD 10, and responsive to that detection, initiate generation of the completed enrollment request and then, automatically submit the completed enrollment request to monitoring service 6 for an immediate enrollment of IMD 10 and commencement of its operation for patient 4.

The first mode may automate operations for the enrollment process, including the generation and/or submission of the completed enrollment request, by having a medical application or another application (e.g., a browser application) running in external device 12 transmit a GUID to the computing service. In one example, external device 12 may transmit the GUID in the completed enrollment request and in return, receive the successful enrollment response; or, as an alternative, external device 12 may transmit the GUID in exchange for attribute information to complete an incomplete enrollment request and generate the completed enrollment request. Under the first mode, external device 12 may receive the GUID through various techniques. In one example, external device 12 may request the GUID (e.g., in a GUID request) from the computing service.

In another example of the first mode, an example GUID request may be a request for an access token. An access token may enable the patient to securely authenticate themselves to monitoring service 6. Monitoring service 6 and/or a third-party system may return token response data including the requested access token. Access tokens enable application 88 to securely call a registration process running on monitoring service 6. In one example, monitoring service 6 may include a computing device hosting an Internet-accessible (e.g., protected) API to perform authentication and authorization for application 88. In one example, application 88 may process a token as an opaque string. Monitoring service 6 may generate the appropriate GUID for facilitating enrollment for patient 4 via their medical application (e.g., and upon medical device implantation). In responding to receiving the access token, monitoring service 6 extracts login credentials and after verifying those login credentials, permits access to sensitive patient information. In another example, the GUID authenticate the user and grant authorization to access that patient account.

As an alternative to requesting the GUID, the first mode may leverage a pre-completed enrollment request that was generated prior to the implantation. In one example, the first mode may leverage a pre-determined GUID that was generated by the computing service and previously established for enrolling the medical device upon implantation. In another example, external device 12 may forward the received data indicative of the implantation.

In another example, application 88 may be configured to parse any token format (e.g., instead of as opaque strings) and auto-generate the appropriate token with the necessary information to complete the enrollment request. Application 88 may include (e.g., amongst its stack of sub-programs) a set of programs for the security platform. Application 88 may implement a universal stack of security programs and by doing so, may use a session cookie to exchange enrolment data. A session cookie may be used given that no token is actually being (e.g., explicitly) exchanged. Application 88 may submit the completed enrollment request via a secure protocol such as SAML. The security platform may employ a number of mechanisms to perform the authentication.

For the second mode, the processing circuitry 80 of external device 12 may detect the implantation of the medical device and automatically generate the completed enrollment request, for instance, for presentation to the patient or another device user. In this context, the completed enrollment request may be characterized as an auto-generated enrollment request. Minor patient action may be required, for instance, to finalize submission of the auto-generated/completed enrollment request, and/or to add a credential for registering a new account for the patient and/or a medical application.

For the third mode, processing circuitry 80 of external device 12 may detect the implantation of the medical device and responsive to that detection, generate a graphical user interface (GUI) component with information notifying the patient of the implantation. The computer display may output, to the GUI, visual indicators prompting the patient to download and/or activate application 88. Processing circuitry 80 of external device 12 may configure the GUI to direct the patient to launch application 88 (e.g., from an App Store property of a smart phone) and then, provide, as input, the GUID generated for the implanted medical device. As further described herein, application 88 may generate a separate GUI to facilitate creation of a new account for the patient, for instance, by submitting a credential for authentication by the computing service.

As described herein, the completed enrollment request denotes a structured dataset having sufficient information for receiving a successful enrollment response from the computing service. For patient 4 and external device 12, the GUID generated by monitoring service 6 for IMD 10 enables automatically generating and then, submitting the completed enrollment request for immediate approval. In one example, processing circuitry 80 of external device 12 may use the GUID to retrieve attributed information to include in the structured dataset, thereby completing the incomplete enrollment request. In one example, processing circuitry 80 of external device 12 may invoke communication circuitry to submit the completed enrollment request to monitoring service 6, successfully completing the enrollment process of IMD 10 for remote monitoring and device support.

In one example, successfully completing the enrollment process with monitoring service 6 provides patient 4 with various forms of support, for instance, health event detection. Monitoring service 6 may be configured with sufficient computing resources capable of running accurate and efficient algorithms for analyzing various patient data for patient 4. Monitoring service 6 may interact with patient 4 via a graphical user interface (GUI) presented on a computer display of external device 12. In one example, processing circuitry 80 of external device 12 may leverage functionality of application 88 to exchange various information with monitoring service 6. In one example, monitoring service 6 may direct patient action by way of visual/audio data for presentation on the GUI and/or gesture recognition via a NUI.

Monitoring service 6 may store data to complete the enrollment process, including mapping data associating the GUID with attributes having sufficient information for satisfying any requirements set forth under monitoring service 6, for example, by using the retrieved attribute information for auto-generating a computerized or electronic document (e.g., form or web form) representative of the completed enrollment request. As one example embodiment of an enrollment request as described herein, this electronic form is configured for presentation on the computer display via the GUI produced by an application (e.g., a client application for monitoring service 6 and/or IMD 10, such as application 88 described herein). The above-mentioned at least one file may include content data that when rendered by the application, may be presented via the GUI. The electronic form may be further configured for interaction with patient 4 by way of GUI components having access to memory buffers for input devices/output devices. Some of these GUI components may be (input) form fields where each form field is presented as a combination of a text block for a description of acceptable input and an input block for a user to enter an example of the acceptable input.

In the example operation of FIG. 6, processing circuitry 80 of external device 12 submits the electronic form with form fields that are pre-populated with appropriate enrollment data (104). This may be accomplished by adding the retrieved attribute information to appropriate form fields of an electronic form representative of a suitable yet incomplete enrollment request and creating a modified electronic form representative of a suitable and completed enrollment request with requisite enrollment data for successfully enrolling into monitoring service 6, which are followed by transmitting the electronic form over a network connection. The above-mentioned application may maintain the network connection as a source/destination for outgoing/incoming data transmissions corresponding to a successful enrollment into remote monitoring and medical device support services provided by computer systems for monitoring service 6.

By submitting the electronic form having pre-populated form fields, processing circuitry 80 of external device 12 can provide monitoring service 6 with sufficient information for completing the enrollment process. In addition to enrolling in a remote monitoring service for detecting health events, monitoring service 6 may create a new patient account and use the enrollment data provided in the electronic form to register that account for patient 4. Application 88 running on external device 12 may be configured as a client application for monitoring service 6 for which access is granted given the virtue of patient 4 being an accountholder. The patient account operates as a credential authorizing the provision, by monitoring service 6, of resource capacities and capabilities related to health event detection.

When in the embodiment of the electronic form, the submission of the completed enrollment request may be accomplished by memorializing the electronic form in at least one stored file and then, transmitting that at least one file in the form of packetized data to a network device running in monitoring service 6. For a number of reasons, monitoring service 6, responsive to the GUID and/or the transmitted electronic form, may forego an examination of the submitted enrollment request for compliance with requirements set forth for enrollment and instead, complete the enrollment process for IMD 10, for example, by returning a successfully enrollment response with minimal latency.

In one example, processing circuitry 80 of external device 12 may recognize an incoming packetized data transmission as data indicative of successful enrollment. Responsive to that data, processing circuitry 80 of external device 12 may execute functionality of application 88, or another medical application associated with IMD 10, for monitoring service 6, or another computing service (106). In one example, processing circuitry 80 of external device 12 may enable a function that when activated, is operative to commence medical device operation of IMD 10, for example, by communicating an instruction via a wireless channel. This instruction may be directed to a destination processor capable of starting/resuming operation. The destination processor may be further configured to control over modifying medical device operational settings; another instruction may be directed to the destination processor for overriding any settings.

The order and flow of the operation illustrated in FIGS. 6 and 7 are examples. In other examples according to this disclosure, more or fewer thresholds may be considered. Further, in some examples, processing circuitry may perform or not perform the methods of FIG. 6 and FIG. 7, or any of the techniques described herein, as directed by a user, e.g., via external device 12 or computing devices 99. For example, a patient, clinician, or other user may turn on or off functionality for identifying changes in patient health (e.g., using Wi-Fi or cellular services) or locally (e.g., using an application provided on a patient's cellular phone or using a medical device programmer).

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

What is claimed is:

1. A computing device comprising:
communication circuitry communicatively coupled to a computing service via a network connection;
processing circuitry; and
memory comprising programming instructions that, when executed by the processing circuitry, cause the processing circuitry to:
responsive to receiving input comprising a globally unique identifier (GUID) corresponding to an application for a patient having an implanted medical device, generate a completed enrollment request for communication, via the communication circuitry, to the computing service to enroll the patient with the computing service, wherein the computing service stores data comprising a mapping between the GUID and attribute information for the completed enrollment request;
receive from the computing service a successful enrollment response based upon the communication of the completed enrollment request; and
execute functionality of the application associated with the implantable medical device and the computing service.

2. The medical system of claim 1, wherein to generate the completed enrollment request, the processing circuitry is further configured to pre-populate form fields of an electronic form representing the completed enrollment request.

3. The medical system of claim 1 further comprising:
a computer display configured to output an electronic form representing an incomplete enrollment request provided by the computing service; and
wherein to generate the completed enrollment request, the processing circuitry is further configured to:
receive the incomplete enrollment request from the computing service; and
transmit the GUID causing the computing service to release enrollment data to form fields of the electronic form causing the computer display to output a pre-populated electronic form representing the completed enrollment request.

4. The medical system of claim 1, wherein to generate the completed enrollment request, the processing circuitry is further configured to communicate, to the computing service, a credential for registration of a new account, wherein the computing service is configured to authenticate the credential.

5. The medical system of claim 1, wherein the processing circuitry is further configured to execute the application to communicate, via the communication circuitry, a GUID request causing the computing service to generate the GUID.

6. The medical system of claim 1, wherein to generate the completed enrollment request, the processing circuitry is further configured to communicate the GUID causing the computing service to return the attribute information for the completed enrollment request.

7. The medical system of claim 1, wherein to generate the completed enrollment request, the processing circuitry is further configured to receive, from the computing service, enrollment data to populate an incomplete enrollment request into the completed enrollment request.

8. The medical system of claim 1 further comprising a mobile computing device of the patient for running the application.

9. The medical system of claim 1, wherein the GUID comprises a credential for the patient, wherein the computing service may authorize the credential for generating the successful enrollment response.

10. The medical system of claim 1, wherein the processing circuitry is further configured to, from the computing service, receive, via the communication circuitry, the GUID that is generated for the implanted medical device.

11. A method of a medical system performed by processing circuitry of a computing device of a patient, the method comprising:
establishing a network connection for coupling communication circuitry of the computing device with a computing service;
responsive to a receiving input comprising a globally unique identifier (GUID) corresponding to an application for a patient having an implanted medical device, generating a completed enrollment request for communication, via the communication circuitry, to the computing service to enroll the patient with the computing service, wherein the computing service stores data comprising a mapping between the GUID and attribute information for the completed enrollment request;
receiving from the computing service a successful enrollment response based upon the communication of the completed enrollment request; and
execute functionality of the application associated with the implantable medical device and the computing service.

12. The method of claim 11, wherein generating the completed enrollment request further comprises:
pre-populating form fields of an electronic form representing the completed enrollment request.

13. The method of claim 11, wherein generating the completed enrollment request further comprises:
outputting, via a computer display, an electronic form representing an incomplete enrollment request, wherein the computing service provides the incomplete enrollment form;
receiving the incomplete enrollment request from the computing service; and
transmitting the GUID to cause the computing service to release enrollment data to form fields of the electronic form;
outputting, via the computer display, a pre-populated electronic form representing the completed enrollment request.

14. The medical system of claim 1, wherein to generate the completed enrollment request, the processing circuitry is further configured to communicate the GUID causing the computing service to return the attribute information for the completed enrollment request.

15. The method of claim 11, wherein generating the completed enrollment request further comprises:
receiving, from the computing service, enrollment data to populate an incomplete enrollment request into the completed enrollment request.

16. The method of claim 11 further comprising establishing a wireless channel to communicatively coupled the patient device with the implanted medical device.

17. The method of claim 11 further comprising:
executing the application to communicate, via the communication circuitry, a GUID request causing the computing service to generate the GUID.

18. The method of claim 11, wherein generating the completed enrollment request further comprises:
communicating the successful enrollment response to the implanted medical device, wherein the successful enrollment response comprises an authorization to commence operation of the implanted medical device.

19. A non-transitory computer-readable storage medium comprising program instructions that, when executed by processing circuitry of a medical device, cause the processing circuitry to:
establish a network connection for coupling communication circuitry of a computing device of a patient with a computing service;
responsive to a receiving input comprising a globally unique identifier (GUID) corresponding to an application for the patient having an implanted medical device, generate a completed enrollment request for communication, via communication circuitry, to a computing service to enroll the patient with the computing service, wherein the computing service stores data comprising a mapping between the GUID and attribute information for the completed enrollment request; and
receive from the computing service a successful enrollment response based upon the communication of the completed enrollment request; and
execute functionality of an application associated with the implantable medical device and the computing service.

20. The non-transitory computer-readable storage medium of claim 19, wherein the instructions that cause the processing circuitry to detect the change in patient health further comprise instructions that cause the processing circuitry to:
pre-populate form fields of an electronic form configured to represent the completed enrollment request.
